(19) United States
(12) Patent Application Publication
(10) Pub. No.: US 2005/0059952 A1
(43) Pub. Date: Mar. 17, 2005

(54) I.V. solution bag with a needleless port

(76) Inventors: Amy S. Giuliano, Sorrento, FL (US); Ralph Gousse, Longwood, FL (US)

Correspondence Address: LITMAN LAW OFFICES, LTD., P.O. BOX 15035 CRYSTAL CITY STATION, ARLINGTON, VA, 22215, US

(21) Appl. No.: 10/663,787

(22) Filed: Sep. 17, 2003

(51) Int. Cl. A61B 19/00

(52) U.S. Cl. 604/408

(57) ABSTRACT

An intravenous (I.V.) solution bag is adapted for receiving fluid materials administered from needleless administering devices, for limiting or preventing contamination resulting form the administration of the fluid materials, and for limiting an amount or volume of fluid material which may become airborne from disengagement of the needleless administering devices from the I.V. solution bag. The I.V. solution bag has a receiving chamber defined therein, at least one access port, and a backflow check valve device secured at the access port. The valve device has a housing and a backflow check valve member, wherein the valve member has an inlet end and a deformable body both disposed within the housing. The inlet end of the valve member is flushed with the inlet end of the housing such that a deformable seal is defined and formed. The I.V. solution bag may further have a handle for hanging the I.V. solution bag.

### BACKGROUND OF THE INVENTION

1. Field of the Invention

The present invention generally relates to intravenous (I.V.) solution bags. More specifically, the present invention relates to an I.V. solution bag adapted for receiving fluid materials from needleless administering devices, for limiting or preventing contamination resulting from the administration of fluid materials, and for limiting an amount or volume of a fluid material which may become airborne from disengagement of a needleless administering device from the I.V. solution bag.

2. Description of the Related Art

A number of valve devices, containers for receiving fluids, and I.V. solution bags have been devised for receiving and/or regulating the reception, mixing, administration, and/or flow of fluids. The classic I.V. solution bag is generally two polymeric or plastic sheets superimposed on each other and sealed together at their periphery defining a receiving chamber therein. The I.V. solution bag generally has an access port adapted for receiving fluid materials from an administering device, such as a syringe, having a needle.

With OSHA pushing the medical field to go to a needle free system for safety and disposal reasons, the present invention helps health professionals to accomplish certain medical tasks, such as mixing medications or administering fluid materials into I.V. solution bags, without the hazards of getting needle sticks. Devices exist to insert into a vial of medication which have a luer lock system on them for pulling up medications, but then the health professional has to put a needle on the syringe and use that needle to administer the medication into an I.V. bag. Health professionals have stuck themselves when administering medications into I.V. bags by the needle going through the rubber stopper at the wrong angle.

For example, U.S. Pat. No. 4,447,230, issued May 8, 1984 to Gula, et al., describes an I.V. administration set assembly which is capable of introducing fluid from a plurality of sources of I.V. fluids into a patient and which allows the various sources of fluid to be easily attached and detached from the assembly without the necessity for intervening safety steps, and yet without any possibility. of air or bacteria being introduced through the system into the patient. An air-eliminating filter and check valves are also described.

U.S. Pat. No. 4,838,875, issued Jun. 13, 1989 to Somor, describes an apparatus for I.V. administration of fluid by injecting same using a needleless syringe directly into the hollow portion of the apparatus through an opening therein. A normally closed backflow check valve device, having a component thereof acting as a means for receiving the fluid by engagement to the syringe, is permanently sealed into the opening in the hollow portion of the apparatus. To protect the opening into the apparatus from contamination, the apparatus is capped by a double luer locking cap. This is accomplished by using a cap structure wherein the cap, which will ultimately re-cover the opening, is locked into and protected by the cap which initially covers the opening, and then discarding this initial cap after the apparatus has been filled.

U.S. Pat. No. 5,059,173, issued Oct. 22, 1991 to Sacco, describes an I.V. administration set-up that includes a main flow line having a capped spike for receiving an I.V. bag at the top end thereof and a needle unit at the bottom end thereof for injecting fluids into a patient. The main flow line has a first drip chamber mounted therein which is capable of administering fluids at a first flow rate.

U.S. Pat. No. 5,439,451, issued Aug. 8, 1995 to Collinson, et al., describes a medical backcheck valve that includes a hollow housing containing an upwardly biased piston assembly for controlling liquid flow therethrough. The piston assembly comprises a substantially rigid plug having one end protruding from the upper end of the housing, and a flexible sheath covering having a relatively small diameter upper end surrounding the plug, a main seal for controlling flow through said first flow space, and a tubular lower end, secured at the bottom of the housing, for preventing liquid from entering within the covering.

U.S. Pat. No. 5,445,630, issued Aug. 29, 1995 to Richmond, describes a hollow spike having a pointed distal end, a proximal end, and a luer fitting positioned in the proximal end of the spike to engage a complementary fitting associated with another IV component. A reflex valve is disposed in the luer fitting to permit fluid flow through the spike when a complementary fitting is connected to the luer fitting of the spike, and to prevent fluid flow through the spike when no fitting is engaged with the fitting of the spike.

U.S. Pat. No. 5,728,086, issued Mar. 17, 1998 to Niedospial, Jr., describes a universal, flexible container with multiple access ports having first and second polymeric sheets sealed together at their periphery defining an interior reservoir, a first access member at the center of the bottom portion equipped with tubing means and one-way her slip stopcock, a needle access member located on one side of the first access member, and a spike access member located on the other side of the first access member.

U.S. Pat. No. 5,782,816 issued on Jul. 21, 1998 to Werschmidt et al., and a web page published by Maximus Medical Products, Inc. titled “A full-line of needlefree products to meet your needs . . . The MaxPlus™ Needleless Connector”. published in July 2003, disclose a bi-directional valve. The valve is a connector adapted to facilitate medicinal access into an intravenous tube that includes a housing having a base and a cap defining a channel through the housing. A valve element is disposed in the channel that includes a plug and a shaft that biases the plug into engagement with the housing.

U.S. Pat. No. 6,063,062, issued May 16, 2000 to Paradis, describes a universal luer activatable and swabable antireflux valve formed by a housing having an inlet and an inwardly biased and circumferentially open flexible seal depending from the inlet to engage and seal a fitting as it enters the inlet. A stationary probe within the housing extends axially from an outlet to the inlet, with the probe having a passageway thereinto connected to the outlet and the input is sealed when there is a reflux of fluid into the outlet.

U.S. Pat. No. 6,068,617, issued May 30, 2000 to Richmond, describes an I.V. port comprising an I.V. container, at least one port body extending from the I.V. container, a valve body operably engaged within the port body and defining a fluid passageway for permitting fluid flow into and out of the I.V. container, and a valve member positioned in the valve body for selectively blocking fluid through the valve body. The valve member defines an outer periphery that is uninterrupted within the periphery, and is disposed in the valve body and biased to a closed configuration, wherein a passageway for fluid communication is not established through the valve body, and is movable to an open configuration when a mating component is engaged, wherein two-way fluid communication through the valve body is permitted.

U.S. Pat. No. 6,106,502, issued Aug. 22, 2000 to Richmond, describes an I.V. set including a drip chamber having a top end and a bottom end. A spikeless connector covers the top end, and an I.V. tube is connected to the bottom end of the drip chamber. The end of the I.V. tube opposite the drip chamber is engaged with a needleless/spikeless connector. Thereby, a needleless/spikeless I.V. set is rendered. A reflux valve including a deformable valve member and reciprocating valve element is also disclosed for use in conjunction with the needleless/spikeless I.V. connectors.

U.S. Pat. No. 6,156,025, issued Dec. 5, 2000 to Niedospial, Jr., et al., describes a twist valve in communication with a pre-filled flexible container and includes a proximal part and a distal part each having a fluid inlet and a fluid outlet. The fluid outlet of the proximal part is blocked with a stopper in the distal part when the twist valve is in the closed position, and is unblocked by the stopper when the twist valve is in the open position.

U.S. Pat. No. 6,299,132, issued Oct. 9, 2001 to Weinheimer, et al., describes a reflux valve engageable with corresponding structure, such as with another corresponding valve or instrument. The reflux valve includes an actuator which is located in, and shiftable in, a valve body. When the corresponding structure is engaged with the actuator, the actuator shifts in the valve body, and pushes a valve stem out of engagement with a valve seat. As a result, liquid is allowed to flow, in one direction, through the actuator, past the valve seat, along an area adjacent a periphery of the valve stem, and out the valve body. Preferably, when the valve stem is unseated from the valve seat, liquid can also flow in an opposite direction.

U.S. patent application Ser. No. 2002/0128628 published on Sep. 12, 2002 to Fathallah, M. A. discloses a drug delivery system. The system stores a beneficial agent and mixes it with a component in a reservoir and then delivers the mixture to a patient.

U.S. patent application Ser. No. 2002/0147440 published on Oct. 10, 2002 to Samolyk, K. A. discloses a method of autologous blood recovery. The invention is directed to a blood bag system comprising a sterile bag of bio-compatible material with an infusion port at the upper end of the bag, an outlet port at the lower end of the bag, a hemo-concentrator, a pair of inlet tubes connected to the hemo-concentrator and a pair of outlet tubes fluidly connected to an outlet port on the hemo-concentrator.

U.S. Pat. No. 6,520,932, issued Feb. 18, 2003 to Taylor, describes an in-line drug delivery pack that connects in-line with an I.V. line and allows for the mixing of diluent with a drug reagent to be delivered to the patient. An internal drug bed bypass mechanism is tailored to apportion diluent flow between the bypass and the drug bed. The apportionment is selected to achieve a solution concentration suitable for I.V. administration as the dried reagent is dissolved.

W095/32748, published Dec. 7, 1995 to Nathan Palestrant, describes a self-sea ling valve device for an angiographic catheter including a housing having a first end adapted to form a luer lock connection with the proximal end of the angiographic catheter. A central bore extends between the first end of the housing and an opposing second end, and a deformable slit seal is supported across the central bore to selectively seal the proximal end of the catheter. The second end of the housing is provided with a female luer lock fitting for receiving the conical tip of a syringe, stopcock, or the like, and forming a pressure tight connection therebetween.

None of the above inventions and patents, taken either singly or in combination, is seen to describe the instant invention as claimed. Thus, an I.V. solution bag adapted for receiving fluid materials administered from needleless administering devices, for limiting or preventing contamination resulting from the administration of fluid materials, and for limiting an amount or volume of a fluid material which may become airborne from disengagement of a needleless administering device from the I.V. solution bag is desired.

### SUMMARY OF THE INVENTION

The present invention is an I.V. solution bag adapted for receiving fluid materials administered from needleless administering devices, for limiting or preventing contamination resulting from the administration of fluid materials, and for limiting an amount or volume of a fluid material which may become airborne from disengagement of a needleless administering device from the I.V. solution bag. The I.V. solution bag defines a receiving chamber therein and has at least one first access port adapted for permitting fluid materials to be administered into or withdrawn from the receiving chamber.

The I.V. solution bag also has a positive pressure valve device secured at the first access port and in fluid communication with the receiving chamber, and may further have a handle adapted for hanging the I.V. solution bag.

The valve device has a housing and a valve member disposed within the housing. The housing has an inlet end, an outlet end and a housing body such that the housing defines a fluid passageway therethrough. The inlet end of the housing is adapted for engagement with outlet ends of the needleless administering devices, and the outlet end of the housing is in fluid communication the receiving chamber.

The valve member has an inlet end and a deformable body, both disposed within the housing. The inlet end of the valve member is flush with the inlet end of the housing such that a deformable seal is defined and formed to limit or prevent contamination from entering the receiving chamber through the fluid passageway. The valve member is biased to a closed configuration wherein the fluid passageway is closed, and is movable to an open configuration wherein the fluid passageway is open to permit fluid materials administered by the needleless administering devices to flow through the fluid passageway.

Accordingly, it is a principal object of the invention to provide an I.V. solution bag adapted for receiving fluid materials administered from needleless administering devices.

It is another object of the invention to provide an I.V. solution bag for limiting or preventing contamination resulting from the administration of fluid materials from needleless administering devices into the I.V. solution bag.

It is a further object of the invention to provide an I.V. solution bag for limiting an amount or volume of a fluid material that may become airborne from disengagement of a needleless administering device from the I.V. solution bag.

It is an object of the invention to provide improved elements and arrangements thereof for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an environmental, frontal view of an intravenous (I.V.) solution bag of the present invention.

FIG. 2 is a perspective, bottom view of the I.V. solution bag of FIG. 1.

FIG. 3 is a perspective, bottom view of the I.V. solution bag of the present invention having another embodiment of the valve device.

FIG. 4 is a perspective view of the valve device of FIG. 1.

FIG. 5 is a cross-sectional view of the valve device taken along 5-5 of FIG. 4.

FIG. 6 is an environmental, cross-sectional view of the valve device of FIG. 5, showing the direction of flow of fluid material through the fluid passageway of the valve device.

Similar reference characters denote corresponding features consistently throughout the attached drawings.

### DETAILED DESCRIPTION OF THE PREFERRED EMBODIMENT

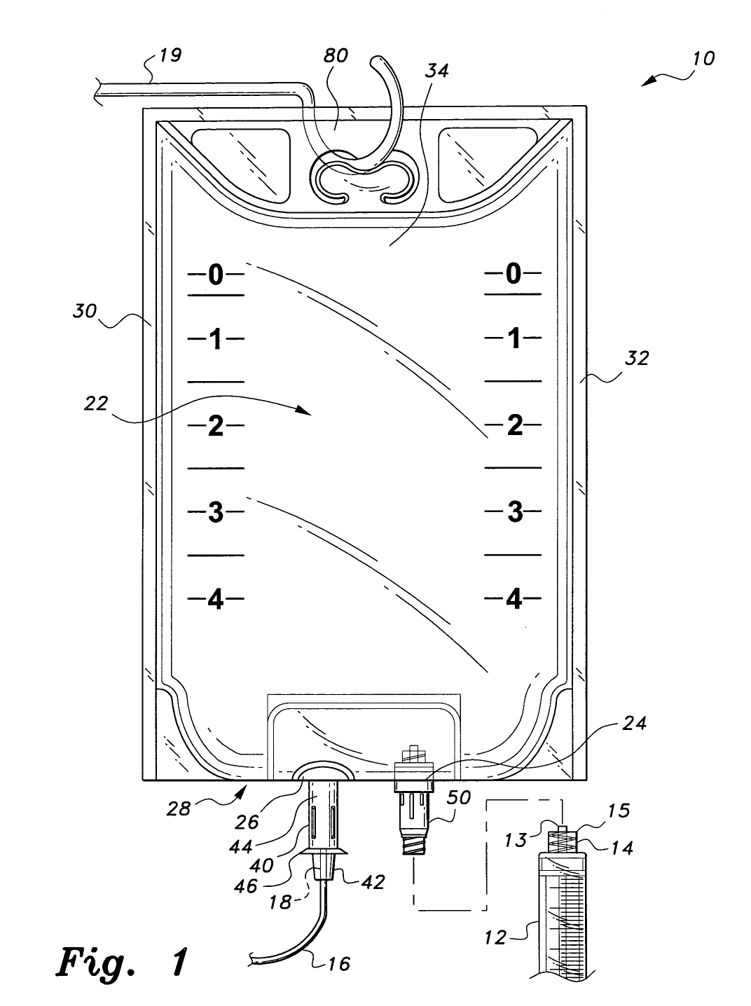

Referring to FIGS. 1-6, the present invention is an intravenous (I.V.) solution bag 10 adapted for receiving fluid materials administered from needleless administering devices, such as a syringe 12 having an outlet end 14 and no needle, for minimizing or preventing contamination resulting from the administration of fluid materials, and for limiting an amount or volume of a fluid material which may become airborne from disengagement of a needleless administering device from the I.V. solution bag 10. The present invention also allows for fluids to be withdrawn from the I.V. solution bag 10, for mixing with powdered medications, with a needleless fluid collection device.

As shown in FIGS. 1-3, the I.V. solution bag 10 has a receiving chamber 22, a first access port 24, a second access port 26, a positive pressure valve device 50 securely sealed at the first access port 24, and a spike access port extension 40 securely sealed at the second access port 26. The I.V. solution bag 10 may further have a handle 80 adapted for hanging the I.V. solution bag 10 upon a secured or standing structure 19.

The I.V. solution bag 10 is generally two polymeric or plastic sheets superimposed on each other and sealed together at their periphery defining a receiving chamber 22 therein. The I.V. solution bag 10 may be a universal, flexible, I.V. solution bag, but may also be a semi-rigid or rigid solution bag.

The I.V. solution bag 10 may be manufactured in many useful sizes, such as 500 ml or any other size known in the art, to receive and/or contain a predetermined volume or amount of I.V. solution, medication in the form of liquids or gels, cell or tissue culture, other fluid materials, and the like. As an example of the usage of the present invention, the I.V. solution bag 10 may be manufactured to contain or store, before usage, a predetermined volume of I.V. solution, and then, when needed or desired, a certain volume or amount of medication may be administered or added to the stored I.V. solution by the syringe 12 through the fluid passageway 68 defined by the housing 60 of the positive pressure valve device 50.

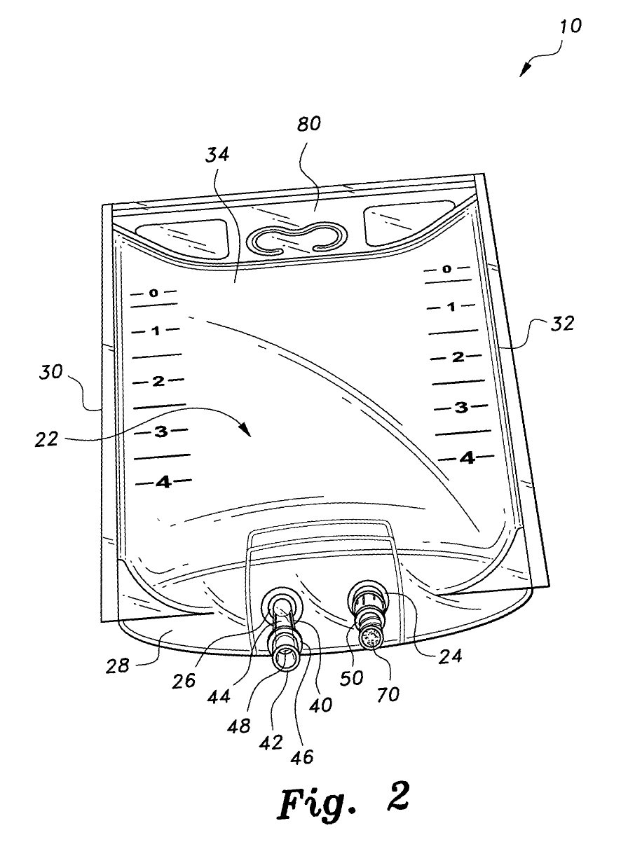

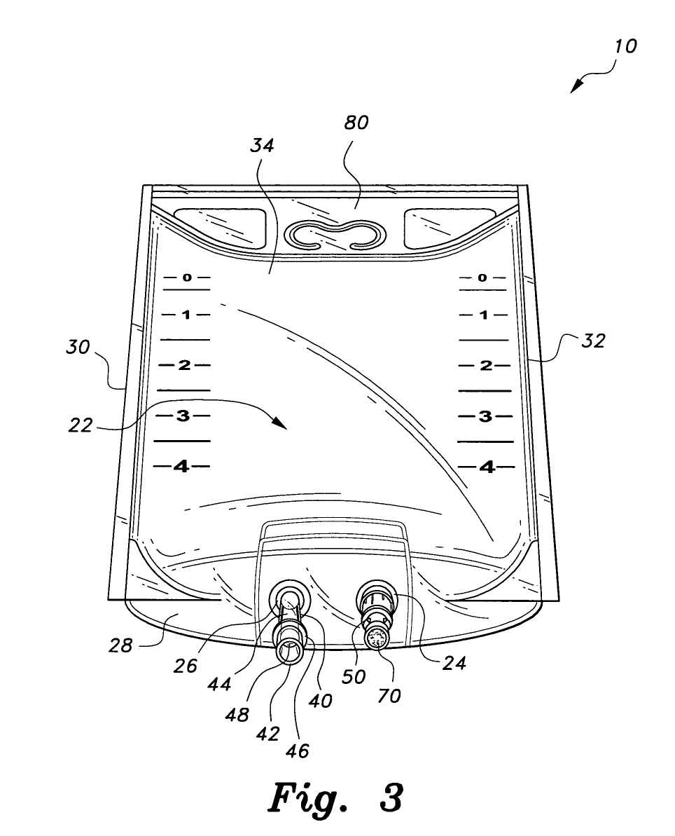

As best shown in FIGS. 2 and 3, the first access port 24 and second access port 26 are located about one another toward the center of the bottom portion 28 of the I.V. solution bag 10. Alternatively, one or both of the access ports 24,26 may be located at a side portion 30,32 and/or top portion 34 of the I.V. solution bag 10.

The spike access port extension 40, which extends downwardly from the bottom portion 28 of the I.V. solution bag 10, is securely sealed at the periphery of the second access port 26. As best shown in FIGS. 2 and 3, the spike access port extension 40 has an inlet end 42, a generally hollow, tubular body 44, an engaging flange 46 located externally between the inlet end 42 and tubular body 44 and extending outwardly from the axis of the spike access port extension 40, and a puncturable membrane or seal 48 located internally between the inlet end 42 and tubular body 44. The spike access port extension 40 is adapted for engagement or attachment with a receiving device, such as an I.V. tubing 16, a catheter, or other receiving devices known in the art. For example and as shown in FIG. 1, an inlet end 18 of the I.V. tubing 16 may be inserted through the inlet end 42 and into the tubular body 44 such that the inlet end 18 pierces and is held there by the puncturable seal 48.

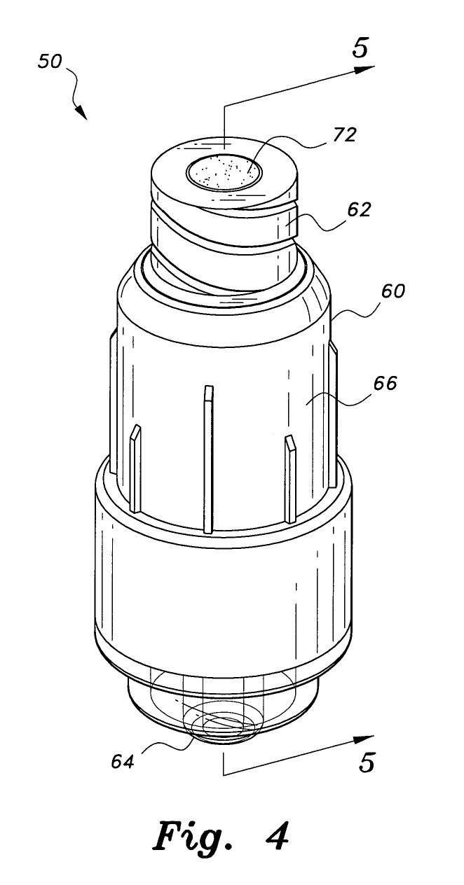

The positive pressure valve device 50, which extends through the first access port 24 and downwardly from the bottom portion 28 of the I.V. solution bag 10, is securely sealed at the periphery of the first access port 24. As best shown in FIGS. 4-6, the valve device 50 has a housing 60 and a valve member 70 disposed within the housing 60. The valve device 50 is in fluid communication with the receiving chamber 22 and fluids contained in the receiving chamber 22 such that the fluid material administered from the syringe 12 will travel through the fluid passageway 68 and into the receiving chamber 22. As shown in detail in FIGS. 4-6, an example of the valve device 50 of the present invention is the MaxPlus™ needleless connector of Maximus Medical Products, Inc. The present valve device 50 is disclosed fully in U.S. Pat. No. 5,782,816 issued to Werschmidt et al. on Jul. 21, 1998, the disclosure of which is hereby incorporated by reference in its entirety. The MaxPlus™ needleless connector has minimal dead space, and is latex-free, DEHP-free and even MRI safe. Also, the MaxPlus™ needleless connector is a positive pressure device that will flush fluid at a specific rate. Alternatively, a backflow check valve device similar to the MaxPlus™ needleless connector and having substantially similar specifications and/or details of the described valve device 50, may be used in the present invention.

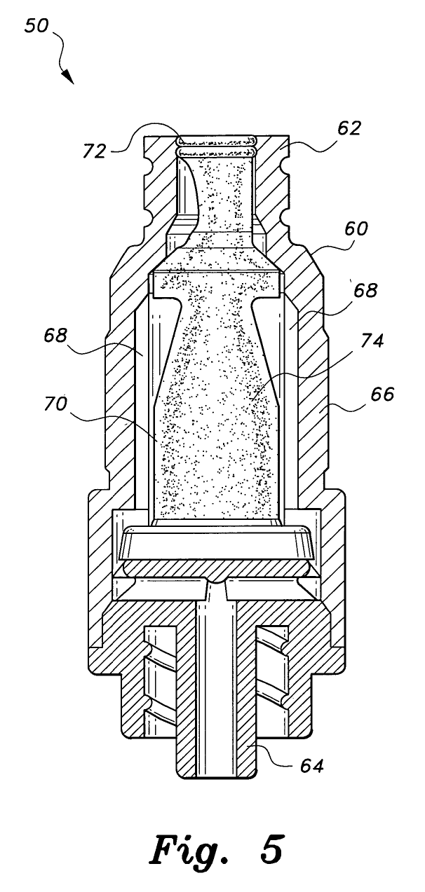

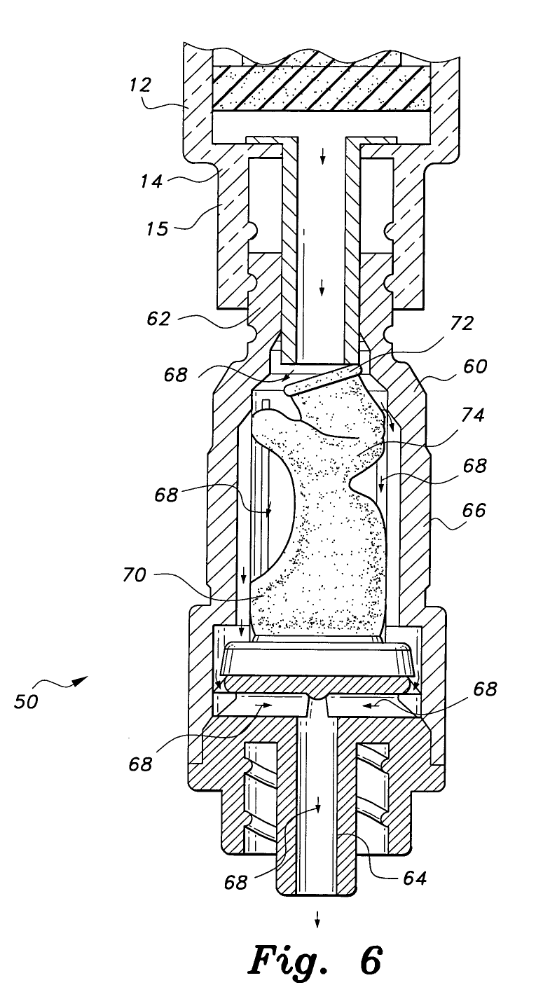

As best shown in FIGS. 5 and 6, the housing 60 has an inlet end 62, an outlet end 64 and a housing body 66 extending from the inlet end 62 to the outlet end 64 of the housing 60 such that the housing 60 defines the fluid passageway 68 the rethrough. The inlet end 62 of the housing 60 is adapted for engagement with the outlet end 14 of the needleless syringe 12, and the outlet end 64 of the housing 60 is in fluid communication with the receiving chamber 22.

The valve member 70 has an inlet end 72 and a deformable body 74 both disposed within the housing 60. The inlet end 72 is flush or substantially flush with the inlet end 62 of the housing 60 such that a deformable seal is defined and formed to limit or prevent contamination from entering the receiving chamber 22 through the fluid passageway 68. If contaminating agents, such as dust, dirt, bacteria, viruses, or other potential contaminating agents, are externally present at or in the vicinity of the deformable seal, the deformable seal can be easily and effectively decontaminated or cleaned by swabbing the seal and the area in the vicinity of the seal with cleaning alcohol or other known cleaners or decontamination agents immediately prior to engaging the outlet end 14 of the needleless syringe 12 to the inlet end 62 of the housing 60. The valve member 70 is biased to a closed configuration such that the fluid passageway 68 is closed from back flow of the fluids and administered fluid material from the receiving chamber 22, and is movable to an open configuration such that the fluid passageway 68 is open to permit the fluid material administered by the needleless syringe 12 to flow through the fluid passageway 68 and to the receiving chamber 22. It is preferred that the valve member 70 is resilient so that the inlet end 72 of the valve member 70 is able to still form a good seal with the inlet end 62 of the housing 60 after more than one administration of the fluid material.

As an example and shown in FIG. 1, the outlet end 14 of the needleless syringe 12 may consist of a centrally located tip 13, and an internally threaded shield 15 surrounding the tip 13 such that the shield 15 is adapted to engage with the correspondingly threaded inlet end 62 of the housing 60. The axial length of the tip 13 is greater than the axial length of the shield 15 such that the tip 13 makes contact with and pushes or moves the deformable body 74 of the valve member 70 to the open configuration when the shield 15 engages with the inlet end 62 of the housing 60. Alternatively, the shield 15 may internally have a female luer fitting, or male luer fitting, adapted to engage with the male luer fitting (as shown in FIG. 3), or female luer fitting, respectively, of the inlet end 62 of the housing 60. It is obvious to one in the art that the outlet end 14 of the needleless syringe 12 and inlet end 62 of the housing 60 may have correspondingly different engaging structures or configurations than the ones described above. It is also obvious to one in the art that the needleless administering device may be an adapter or connector that is connected, attached or secured to a syringe or device that can administer fluid materials.

As shown in FIGS. 1-3, the handle 80 is integrally sealed to the top portion 34 of the I.V. solution bag 20, and is adapted for hanging the receiving apparatus 10 upon the secured or standing structure 19.

For simplicity and economical reasons, the I.V. solution bag 10 may be manufactured as a single unit. Also, the I.V. solution bag 10 may be manufactured of plastic, a polymeric material, and/or any other material known in the art. The valve device 50 may be manufactured by a molding or extrusion process.

It is to be understood that the present invention is not limited to the embodiment described above, but encompasses any and all embodiments within the scope of the following claims.

1. An intravenous solution bag adapted for receiving a fluid material administered from a needleless administering device having an outlet end, comprising:
a flexible bag defining a receiving chamber;
an access port defined in the bag adapted for permitting the fluid material to be administered into said receiving chamber, the access port including a valve having a housing defining an inlet and an outlet and a having a compressible valve member, the outlet opening into the receiving chamber; and
an outlet port adapted for releasing fluid from the receiving chamber through intravenous tubing;
wherein the inlet end of the valve housing is adapted for engagement with the outlet end of the needleless administering device; and
wherein the valve has a closed position in which the compressible valve member is seated flush with the inlet end of the valve housing and forms a seal to limit or prevent contamination from entering said receiving chamber through said fluid passageway, and an open position in which the compressible valve member is compressed into the housing by the outlet end of the needleless administering device, breaking the seal in order to permit the fluid material administered by the needleless administering device to flow through the fluid passageway.

2. The I.V. solution bag of claim 1, wherein said access port is located at a bottom portion of said I.V. solution bag.

3. The I.V. solution bag of claim 1, further comprising a handle attached to said flexible bag adapted for hanging the I.V. solution bag upon a structure.

4. The I.V. solution bag of claim 1, further comprising an extension secured about said outlet port and in fluid communication with said receiving chamber, the extension having an inlet end, a generally hollow, tubular body, an engaging flange located externally between said inlet end and said tubular body and extending outwardly from an axis of the extension, and a puncturable seal located internally between said inlet end and said tubular body, the extension extending outwardly from the flexible bag and being adapted for engaging with a receiving device.

5. The I.V. solution bag of claim 1, wherein said outlet port is a spike access port.

6. The I.V. solution bag of claim 1, wherein said outlet port is located at a bottom portion of said flexible bag.

7. The I.V. solution bag of claim 1, wherein the inlet end of the valve housing is externally threaded.

8. The I.V. solution bag of claim 1, wherein the inlet end of said valve housing further comprises an external luer lok fitting configuration.

9. The I.V. solution bag of claim 8, wherein said luer fitting configuration is a male luer lok fitting.

10. The I.V. solution bag of claim 8, wherein said luer fitting configuration is a female luer fitting.

11. The I.V. solution bag of claim 1, wherein said compressible valve member is resilient.

12. The I.V. solution bag of claim 1, wherein the I.V. solution bag is manufactured as a single unit.
